(19) United States
(12) Patent Application Publication
(10) Pub. No.: US 2026/0097089 A1
(43) Pub. Date: Apr. 9, 2026

(54) COMPOSITION FOR IMPROVING INTESTINAL HEALTH, OR PREVENTING, IMPROVING OR TREATING LEAKY GUT SYNDROME OR METABOLIC DISEASES CONTAINING PARSNIP EXTRACT AS AN ACTIVE INGREDIENT

(71) Applicant: KOREA FOOD RESEARCH INSTITUTE, Jeollabuk-do (KR)

(72) Inventors: Ho-Young PARK, Jeonbuk-do (KR); Ji Eun Park, Jeonbuk-do (KR); Miri Park, Jeonbuk-do (KR); Hee-Kyoung Son, Gwangju (KR); Yu Ra Lee, Jeonbuk-do (KR); Guijae Yoo, Jeonbuk-do (KR); Sang-Yoon Choi, Seoul (KR); Yoon-sook Kim, Jeonbuk-do (KR); Sang Keun Ha, Daejeon (KR); Donghwan Kim, Jeonbuk-do (KR)

(21) Appl. No.: 19/347,064

(22) Filed: Oct. 1, 2025

(30) Foreign Application Priority Data
Oct. 4, 2024 (KR) 10-2024-0135101
Oct. 4, 2024 (KR) 10-2024-0135115

(51) Int. Cl. A61K 36/23; A61P 1/00; A61P 1/16; A61P 3/04; A61P 3/06; A61P 3/10

(52) U.S. Cl. CPC A61K 36/23; A61P 1/00; A61P 1/16; A61P 3/04; A61P 3/06; A61P 3/10; A61K 2236/15; A61K 2236/17; A61K 2236/331

(57) ABSTRACT

The present disclosure relates to a composition containing parsnip extract as an active ingredient which reduces the downregulation of tight junction protein expression, promote the proliferation of beneficial intestinal bacteria, improve the intestinal environment such as maintaining a slightly acidic intestinal environment, and have effects such as reducing intestinal permeability. Therefore, the composition may improve intestinal health and prevent, treat, or improve leaky gut syndrome, and may be used as a material for foods, feeds, pharmaceuticals, or quasi-drugs. Additionally, the composition may have body fat reduction and weight loss activities, reduce the protein expression of lipid metabolism-related factors such as ACC, FAS, SREBP1, ChREBP, G6PD, or C/EBPα, and have effects such as reducing liver weight, reducing blood sugar, reducing ALT or AST levels, reducing insulin resistance, and reducing extent of liver damage. Therefore, the composition may prevent, treat, or improve obesity or a metabolic disease.

### CROSS-REFERENCE TO RELATED APPLICATION(S)

This application claims benefit of priority to Korean Patent Application No. 10-2024-0135101 filed on Oct. 4, 2024, and Korean Patent Application No. 10-2024-0135115 filed on Oct. 4, 2024 in the Korean Intellectual Property Office, the disclosure of which is incorporated herein by reference in its entirety.

### FIELD

The present disclosure relates to a composition containing parsnip extract as an active ingredient for improving intestinal health, or preventing, improving, or treating leaky gut syndrome or a metabolic disease.

### BACKGROUND

The intestines, which are one of the major organs constituting the digestive system, are divided into the small intestine and the large intestine. Food consumed is broken down into nutrients and absorbed in the small intestine, while unabsorbed food residues are excreted through the large intestine. In addition to its functions of digestion, absorption, and excretion of food, the intestines have a close and important relationship with immunity, as approximately 70% of the body's total immune cells are concentrated there. They are continuously subjected to damage from mechanical stress, chemical stress such as carcinogens from the external environment, and biological stress from harmful intestinal bacteria, which negatively affects the intestinal epithelial cells and the production of mucus, thereby threatening intestinal health. In particular, due to improper dietary and lifestyle habits such as modern people's Westernized eating habits, lifestyle-related stress, convenience foods, and excessive drinking and smoking, the number of patients with intestinal diseases has been increasing, and many metabolic diseases, and degenerative brain disease such as diabetes, Parkinson's disease, and Alzheimer's disease, have been reported to be ultimately associated with intestinal health, thereby highlighting the importance of intestinal health for modern people.

Meanwhile, the intestinal epithelial barrier is known to play a crucial role as the first barrier against toxic substances, pathogens, and antigens. In particular, inter-epithelial tight junctions, which contain membrane proteins such as Zonula occludens-1 (ZO-1), occludin, and claudin-1, are essential for maintaining the integrity of the intestinal mucosa by preventing bacteria, toxins, and other harmful substances from penetrating the intestinal barrier. Intestinal mucosal cells maintain a constant intercellular gap, but during the process of digestion and absorption, upon stimulation or injury, a phenomenon occurs in which intestinal mucosal permeability increases, allowing macromolecule substances to pass back and forth through the spaces between cells. When the intestinal barrier fails to function properly, leakage of macromolecule substances from the blood into the intestinal lumen or direct influx of macromolecule substances present in the lumen into the bloodstream may occur, which is referred to as a “leaky gut” condition. The symptoms that appear during this time are collectively referred to as leaky gut syndrome (LGS). This condition can manifest as a variety of clinical conditions such as aging, allergies, multiple trauma, rheumatoid arthritis, inflammatory bowel disease, chronic fatigue syndrome, and irritable bowel syndrome. In addition, increased intestinal mucosal permeability or damage to the intestinal mucosa allows pathogens, antigens, putrefactive substances, and the like to penetrate the intestinal mucosa, thereby inducing inflammatory responses. Endotoxins enter the bloodstream, leading to bacterial translocation, intestinal endotoxemia, and the like, such that various inflammatory and immune responses occur. Therefore, protecting tight junction proteins to maintain the integrity of the intestinal barrier is a very important factor for overall intestinal health.

In addition, numerous microorganisms inhabit the human gastrointestinal tract in the form of communities, collectively referred to as the enteric microbiota. Alterations in the composition and function of the enteric microbiota, leading to intestinal dysbiosis, are known to directly or indirectly influence various diseases, such as autoimmune disorders, inflammatory bowel disease, and obesity.

Accordingly, there is a need to develop excellent new materials for improving intestinal health or ameliorating leaky gut syndrome, which exert activities such as enhancing the barrier function of tight junction proteins and protecting the intestinal mucosa, and promoting the proliferation of beneficial intestinal bacteria while inhibiting the proliferation of harmful intestinal bacteria to maintain enteric microbiota, thereby preventing or ameliorating dysbiosis and improving the intestinal environment, without side effects.

Meanwhile, due to recent rapid economic growth and Westernized lifestyles, significant changes have also occurred in eating habits. In particular, busy modern people are increasingly experiencing metabolic diseases due to the consumption of high-fat diets such as fast food, high-sugar foods, and a lack of physical activity. A metabolic disease is a general term for disorders caused by abnormalities in the body's metabolism, with obesity, diabetes, and the like being representative examples.

Obesity is a chronic disease in which abnormal energy balance regulation or hypernutrition leads to excessive accumulation of adipose tissue, thereby increasing the risk of morbidity and mortality from various diseases. Obesity and its related diseases are a common and serious public health problem in the United States and worldwide. According to the World Health Organization (WHO), it is estimated that one billion or more adults worldwide are overweight, and at least 300 million of them are clinically obese. Excessive lipid accumulation is caused by an imbalance in the expression of genes responsible for lipid oxidation and synthesis in adipocytes, which is attributable to unbalanced lifestyle habits, particularly eating habits. Moreover, excessive lipid accumulation can lead to dysfunction of metabolic regulatory cells, including hepatocytes, thereby contributing to the development of metabolic disorders such as non-alcoholic fatty liver disease. The expression of transcription factors, such as CCAAT/enhancer-binding protein (C/EBP) and peroxisome proliferator-activated receptor-γ (PPARγ), which promote differentiation of adipocytes, is increased in metabolic diseases such as obesity. This transcriptional network regulates the expression of genes involved in fatty acid synthesis and transport, leading to triglyceride accumulation. Furthermore, adipocytes secrete various adipokines, including adiponectin, which can impair insulin sensitivity and may lead to hyperglycemia and insulin resistance.

Such lipid accumulation disrupts tissue function and contributes to the development of obesity-related comorbidities, including hypertension, fatty liver disease, and cardiovascular disease. The economic impact of obesity encompasses both direct and indirect costs, including increased medical expenses and reduced socio-economic productivity, and these burdens continue to rise as the obese population steadily grows. However, despite the increasing prevalence of obesity, there are few effective treatments available. Lifestyle habit modifications such as dietary control and exercise provide only partial benefits. Since maintaining healthy eating habits is considered a safe strategy for preventing obesity, there is a need to identify materials capable of inhibiting lipid accumulation and preventing metabolic diseases.

### SUMMARY

An object of the present disclosure is to solve the above-mentioned problems and other problems associated therewith.

An aspect of the present disclosure is to provide a food composition comprising parsnip extract as an active ingredient for improving intestinal health, or preventing or improving leaky gut syndrome or a metabolic disease.

Another aspect of the present disclosure is to provide a health functional food comprising the food composition described above.

Another aspect of the present disclosure is to provide a pharmaceutical composition comprising parsnip extract as an active ingredient for preventing or treating leaky gut syndrome or a metabolic disease.

Another aspect of the present disclosure is to provide a quasi-drug composition comprising parsnip extract as an active ingredient for preventing or improving leaky gut syndrome or a metabolic disease.

Another aspect of the present disclosure is to provide a feed composition comprising parsnip extract as an active ingredient for improving intestinal health, or preventing or improving leaky gut syndrome or a metabolic disease.

The technical challenges to be achieved according to the technical idea of the present disclosure disclosed in the present specification are not limited to the challenges for solving the problems described above, and other challenges not mentioned can be clearly understood by those skilled in the art from the following description.

This will be specifically described as follows. Meanwhile, each of the descriptions and embodiments disclosed in the present application may also be applied to each other descriptions and embodiments. That is, all combinations of various elements disclosed in the present application fall within the scope of the present application. In addition, the scope of the present application shall not be considered limited by the specific description set forth below.

As one aspect for achieving the above objects, the present disclosure provides a food composition containing parsnip extract as an active ingredient for improving intestinal health, or preventing or improving leaky gut syndrome or a metabolic disease.

As used herein, the term “parsnip (Pastinaca sativa)” refers to a root vegetable of the Apiaceae family, also known as “sugar carrot,” native to Europe and Siberia, having a flavor and taste intermediate between those of carrot and ginseng.

As used herein, the term “extract” encompasses an extract solution itself as well as any formulation derived therefrom, including an extract solution obtained by extracting parsnip, a diluted or concentrated solution of the extract solution, a dried product obtained by drying the extract solution, a processed or purified product of the extract solution, or a mixture thereof.

In addition, the parsnip extract according to the present disclosure is not limited to any specific part of the plant and may be obtained, for example, from the root, flower, leaf, or stem, and is preferably obtained from the root.

The method for extracting the extract of the present disclosure is not particularly limited and may be performed using any method commonly used in the art. Non-limiting examples of the extraction method include hot water extraction, cold maceration, solvent extraction, steam distillation, elution, pressing, ultrasonic extraction, filtration, reflux extraction, and reflux-cooling extraction. These methods may be performed individually or in combination of two or more methods.

The extract of the present disclosure is obtained from parsnip using an appropriate solvent, and may include, for example, crude extracts, extracts soluble in polar solvents, and extracts soluble in non-polar solvents. The type of extraction solvent used to prepare the extract is not particularly limited, and any solvent known in the art may be used. Non-limiting examples of the extraction solvent include water; lower alcohols having 1 to 4 carbon atoms such as methanol, ethanol, propanol, isopropanol, butanol, propyl alcohol, or butyl alcohol; polyhydric alcohols such as glycerin, butylene glycol, or propylene glycol; hydrocarbon-based solvents such as methyl acetate, ethyl acetate, acetone, benzene, hexane, diethyl ether, or dichloromethane; or a mixture thereof. Specifically, the extraction solvent may be water. In addition, a solvent extract may be prepared by extracting one or more times with the solvent.

Specifically, the parsnip extract of the present disclosure may be obtained by drying and grinding parsnip roots, adding water in a volume corresponding to 10 to 30 times the weight of the parsnip roots, and repeatedly performing reflux extraction at a temperature of 60 to 100° C. for 1 to 5 hours per cycle. More specifically, the extract may be obtained by drying and grinding parsnip roots, adding water in a volume corresponding to 20 times the weight of the parsnip roots, and performing reflux extraction at 80° C. for 3 hours.

The extract of the present disclosure may be used as is after removing suspended solid particles by filtration, for example, by filtering out particles using nylon, etc., or by filtering using filter paper or cryofiltration, or may be used after concentration or drying (e.g., freeze-drying, hot-air drying, spray-drying, etc.). Furthermore, the extract may additionally undergo conventional fractionation processes, and may also be purified using conventional purification methods.

As used herein, the term “active ingredient” refers to a component that exhibits the intended activity either alone or in combination with a carrier that is itself inactive.

The active ingredient in the composition of the present disclosure may be included in any amount (effective amount), insofar as it can exhibit activity for improving intestinal health, or preventing, improving, or treating leaky gut syndrome or a metabolic disease, depending on the specific use, formulation, purpose of combination, etc. A typical effective amount may range from 0.000001 wt % to 99.9 wt %, based on the total weight of the composition. The term “effective amount” refers to the amount of the active ingredient included in the composition of the present disclosure that, when the composition of the present disclosure is administered to an individual to whom the composition of the present disclosure is applied for an administration period recommended by those skilled in the art, is capable of exhibiting the intended functional or pharmacological effects, such as improving intestinal health, or preventing, improving, or treating leaky gut syndrome or a metabolic disease. Such an effective amount may be determined by routine experimentation by those skilled in the art.

As used herein, the term “intestinal health” refers to a state where intestinal functions are normally maintained or improved, or where prevention or treatment of various intestinal diseases is achieved.

As used herein, the term “intestinal function” refers to the entirety of functions performed by the intestine in the life activities of an organism. For example, such intestinal functions include, but are not limited to, the digestive functions for the intake and decomposition of external substances, the function of supplying nutrients through absorption of substances decomposed by digestion, excretory functions, such as elimination of bodily wastes through defecation and urination, and the immune functions for protecting the body from external substances entering the intestine.

As used herein, the term “improvement of intestinal function” refers to promoting the smooth execution of intestinal functions of an organism or increasing the efficiency thereof. For example, improvement of intestinal function may include enhancement of intestinal immune function through increased mucin production covering the intestinal epithelium, increase in the ratio of beneficial bacteria in the enteric microbiota, reduction in the proportion of harmful bacteria, balance maintenance of enteric microbiota, and correction of dysbiosis.

As used herein, the term “enteric microbiota” refers to the complex microbial community inhabiting the intestinal tract. This microbial community, the enteric microbiota, may affect intestinal function through activities such as decomposition or secretion of substances within the intestine. The enteric microbiota includes beneficial bacteria that exert favorable effects on the intestine, such as promoting intestinal motility, enhancing intestinal immunity, and suppressing the growth of harmful bacteria, as well as harmful bacteria that induce constipation, diarrhea, and inflammatory bowel diseases.

Specifically, the parsnip extract of the present disclosure may aid in formation of the intestinal mucosa by reducing the downregulation of tight junction proteins expression, and may prevent an increase in permeability of harmful substances from colon tissues, etc.

In the present disclosure, the “tight junction protein” may be Zonula occludens-1 (ZO-1), but is not limited thereto.

In addition, in the present disclosure, the composition may improve the intestinal environment, specifically by promoting the proliferation of beneficial intestinal bacteria and reducing the proliferation of harmful intestinal bacteria, thereby improving the intestinal environment.

In the present disclosure, the “beneficial intestinal bacteria” may be one or more selected from the group consisting of Lactobacillus bulgaricus, Lactobacillus gasseri, Lactobacillus acidophilus, Lactobacillus delbrueckii subsp. bulgaricus, Lactobacillus helveticus, Lactobacillus fermentum, Lactobacillus paracasei, Lactobacillus plantarum, Lactobacillus reuteri, Lactobacillus rhamnosus, Lactobacillus salivarius, Lactococcus lactis, Streptococcus thermophilus, Bifidobacterium bifidum, Bifidobacterium breve, Bifidobacterium animalis, and Bifidobacterium longum, and the “harmful intestinal bacteria” may include one or more selected from the group consisting of Escherichia coli, Shigella flexneri, Salmonella typhimurium, Enterobacter aerogenes, Enterococcus faecalis, and Staphylococcus aureus, but is not limited thereto.

In the present disclosure, the composition may inhibit the proliferation of harmful intestinal bacteria and improve the intestinal environment by maintaining the intestinal environment at a slightly acidic pH.

In the present disclosure, the composition may prevent or improve leaky gut syndrome by reducing intestinal permeability.

In one embodiment of the present disclosure, it was confirmed that the administration of the parsnip extract led to a decrease in pH in the culture of Lactobacillus bulgaricus, Lactobacillus gasseri, Bifidobacterium bifidum, and Bifidobacterium longum strains, and this decrease was more pronounced compared to the positive control group, inulin, which is known to improve the enteric microbiota.

Furthermore, it was confirmed that in one embodiment of the present disclosure, for the intestinal mucosa, changes in the expression of the tight junction protein Zonula occludens-1 (ZO-1) was observed, and the parsnip extract-treated groups effectively reduced the downregulation of the tight junction protein expression.

In addition, it was confirmed that in one embodiment of the present disclosure, for the improvement of leaky gut syndrome, mice that were fed a high-fat diet and administered the parsnip extract showed a reduction in the detection concentration of fluorescein isothiocyanate (FITC)-dextran, indicating a significant suppression of intestinal permeability. In the present disclosure, a high-fat diet mouse animal model established by feeding a high-fat diet (60% kcal from fat) has recently been commonly used in studies on intestinal health following the advent of microbiome technologies that induces intestinal dysbiosis.

As used herein, the term “metabolic disease” is a general term for diseases caused by metabolic disorders in the body, and may include, but is not limited to, obesity, diabetes, hyperlipidemia, hypertension, hypercholesterolemia, hyperinsulinemia, arteriosclerosis, dyslipidemia, hypertriglyceridemia, liver disease, fatty liver, stroke, myocardial infarction, cardiovascular disease, hyperglycemia, or insulin resistance. In the present disclosure, the fatty liver disease may be non-alcoholic fatty liver disease, and the non-alcoholic fatty liver disease may be selected from the group consisting of simple fatty liver disease, nutritional fatty liver disease, starvation-induced fatty liver disease, obesity-related fatty liver disease, diabetic fatty liver disease, steatohepatitis, liver fibrosis, and cirrhosis, but is not limited thereto.

The composition of the present disclosure may regulate lipid accumulation by modulating the expression of lipid metabolism regulatory factors. Specifically, the composition of the present disclosure may suppress the protein expression of acetyl-CoA carboxylase (ACC), fatty acid synthase (FAS), sterol regulatory element-binding protein 1 (SREBP1), carbohydrate response element-binding protein (ChREBP), glucose-6-phosphate dehydrogenase (G6PD), and CCAAT/enhancer-binding protein a (C/EBPα), thereby suppressing lipid accumulation and exhibiting preventive, improvement, or therapeutic effects against obesity and metabolic diseases.

As used herein, the term “obesity” refers to a condition of excessive adipose tissue in the body, and may further encompass various complications arising from obesity.

The composition of the present disclosure may suppress lipid accumulation, specifically, intracellular lipid accumulation, and may further exhibit activities such as improving blood lipid levels, reducing body fat, or reducing body weight. Furthermore, the composition of the present disclosure may exhibit metabolic syndrome-suppressing activities, including but not limited to, reducing liver weight, reducing blood glucose, reducing serum levels of alanine aminotransferase (ALT) or aspartate aminotransferase (AST), reducing total cholesterol and triglyceride levels, reducing insulin levels and insulin resistance, reducing serum leptin hormone levels, reducing the extent of liver damage, and reducing epididymal fat size.

As used herein, the term “lipid” is a general term for substances among the components of living organisms that are soluble in organic solvents, and may include, for example, triglycerides, cholesterol, phospholipids, free fatty acids, or combinations thereof.

As used herein, the term “metabolic syndrome” refers to a condition in which symptoms such as elevated blood pressure, hyperglycemia, hypertriglyceridemia, and abdominal obesity occur simultaneously due to abnormal metabolism in the body. This term is a distinct from “metabolic diseases,” which refers to diabetes, hypertension, hyperlipidemia, fatty liver, obesity, and the like, caused by abnormal metabolism in the body.

As used herein, the term “prevention” refers to any action that suppresses or delays the onset of symptoms of leaky gut syndrome or a metabolic disease through administration of the composition of the present disclosure. As used herein, the term “improvement” refers to the effect of promoting intestinal health, or alleviating leaky gut syndrome or a metabolic disease, and their symptoms through application of the composition of the present disclosure.

The food composition of the present disclosure may further include, in addition to parsnip extract, any compound or natural extract that has already been confirmed to be safe and is known to exhibit the corresponding activity in the art, in order to enhance convenience of administration or ingestion.

These compounds or extracts may include compounds or extracts listed in pharmacopeias of various countries (the “Korean Pharmacopoeia” in Korea) and monographs of health functional foods in various countries (“Standards and Specifications for Health Functional Foods” announced by the Ministry of Food and Drug Safety in Korea), compounds or extracts approved for use in accordance with the laws of various countries regulating the manufacture and sale of pharmaceuticals (the “Pharmaceutical Affairs Act” in Korea), or compounds or extracts whose functionality is recognized in accordance with the laws of various countries regulating the manufacture and sale of health functional foods (e.g., the “Health Functional Food Act” in Korea).

As used herein, the term “food” includes meat, sausages, bread, chocolate, candies, snacks, confectioneries, pizza, ramen, other noodles, gum, dairy products including ice cream, various soups, beverages, tea, drink preparations, alcoholic beverages, vitamin complexes, nutritional supplements, functional foods, food additives, health functional foods, and health foods, encompassing all foods in the conventional sense.

The term “health food” refers to a food that provides greater benefits for maintaining or promoting health compared to general food, while the term “health supplement food” refers to a food intended for health-support purposes. In some cases, the terms “health functional food,” “health food,” and “health supplement food” may be used interchangeably.

As used herein, the term “health functional food” is synonymous with food for special health use (FoSHU), and refers to a food processed to efficiently express biological regulatory functions in addition to providing nutrition, thus having high medicinal and medical effects. Here, the term “functionality” refers to obtaining beneficial effects for health purposes, such as modulation of nutrients or physiological actions with respect to the structure and function of the human body. The food of the present disclosure may be prepared by methods commonly used in the art, and during its preparation, raw materials and components commonly added in the art may be incorporated. In addition, the formulation of the food is not limited, as long as it is recognized as food. The food composition of the present disclosure may be prepared in various forms of formulations, and unlike conventional pharmaceuticals, is made from food ingredients and therefore has the advantage of being free from side effects that may occur with long-term use of pharmaceuticals. In addition, due to its excellent portability, the food of the present disclosure may be consumed as a supplement to improve intestinal health or to prevent or improve leaky gut syndrome or a metabolic disease.

Specifically, the health functional food refers to a food product prepared by adding the parsnip extract into food materials such as beverages, teas, spices, gums, or confectioneries, or by formulating it into pills, tablets, capsules, powders, suspensions, etc., which, when consumed, provide specific health benefits. However, the health functional food, unlike conventional pharmaceuticals, is made from food ingredients and therefore has the advantage of being free from side effects that may occur with long-term use of pharmaceuticals.

In addition, the nutritional supplements may be prepared by adding the active ingredient to capsules, tablets, pills, etc. Furthermore, the health functional food is not limited to these forms, and may, for example, be prepared as tea, juice, or drinks and consumed as a health beverage in liquid, granule, capsule, or powder form. Additionally, it may be prepared in the form of a composition by mixing with one or more known active ingredients known to be effective in improving intestinal health, or preventing or improving leaky gut syndrome or metabolic diseases.

In addition to those mentioned above, the health functional food of the present disclosure may contain various nutritional supplements, vitamins, electrolytes, flavoring agents, coloring agents, pectic acids or salts thereof, alginic acid or salts thereof, organic acids, protective colloid thickeners, pH adjusters, stabilizers, preservatives, glycerin, alcohol, or carbonating agents.

The food composition of the present disclosure may be routinely ingested, and is therefore highly useful, as prevention or improvement of intestinal health, leaky gut syndrome, or metabolic diseases may be expected.

The food composition of the present disclosure may be prepared in any form. For example, it may be prepared as beverages such as tea, juice, carbonated drinks, and sports drinks; processed dairy products such as milk or yogurt, food products such as gum, rice cakes, Korean traditional confectionery, bread, confectionery, and noodles, and preparations such as tablets, capsules, pills, granules, liquids, powders, flakes, pastes, syrups, gels, jellies, and bars. In addition, the food composition of the present disclosure may assume any product classification in terms of legal or functional categorization, as long as it complies with the laws and regulations in force at the time of manufacture and distribution. For example, it may be classified as a health functional food under the Korean “Health Functional Food Act,” or as a food type such as confectionery, tea, beverages, foods for special medical purposes, or foods for special dietary use under the food standards (Notification on “Standards and Specifications for Foods” announced by the Ministry of Food and Drug Safety) pursuant to the Korean “Food Sanitation Act.”

In addition to the active ingredients, the food composition of the present disclosure may further include food additives. Food additives may be generally understood as substances that are added to, or mixed with, or permeated into food during its preparation, processing, or preservation, and their safety should be ensured since they are consumed daily and over a long period of time together with food. These food additives are categorized into sweeteners, flavor enhancers, preservatives, emulsifiers, and flavoring agents based on their use.

The sweeteners may be natural or synthetic substances and are used to impart appropriate sweetness to the food. For example, the sweeteners include neotame, lactitol, D-ribose, mannitol, D-maltitol, and sodium saccharin.

The flavor enhancers may be natural or synthetic substances and are used to improve the taste or aroma of food. For example, the flavor enhancers include disodium 5′-guanylate, L-glutamic acid, glycine, and betaine.

The preservatives may include sodium metabisulfite, sulfurous anhydride, sorbic acid, benzoic acid, and grapefruit seed extract.

The emulsifiers are food additives that homogeneously mix or maintain two or more immiscible phases, such as water and oil. Examples thereof include sodium gluconate, glycerol fatty acid esters, lecithin, magnesium stearate, and alginic acid.

The flavoring agents are food additives that impart a unique aroma to foods or restore the original aroma of foods lost during the preparation process. Examples thereof include ethyl vanillin, ethyl octanoate, linalyl acetate, allyl caproate, and para-methylacetophenone.

In addition to the aforementioned food additives, the food composition of the present disclosure may include physiologically active substances or minerals, which are known in the art and recognized as safe food additives, for the purpose of supplementing and enhancing functionality and nutritional value. Examples of physiologically active substances include catechins included in green tea, etc., vitamins such as vitamin B1, vitamin C, vitamin E, and vitamin B12, tocopherols, and dibenzoyl thiamine. Examples of minerals include calcium preparations such as calcium citrate, magnesium preparations such as magnesium stearate, iron preparations such as ferric citrate, chromium chloride, potassium iodide, selenium, germanium, vanadium, and zinc.

The food composition of the present disclosure may include the aforementioned food additives in an appropriate amount to achieve the intended purpose according to the product type. With respect to other food additives that may be included in the food composition of the present disclosure, reference may be made to the food standards or food additive standards of various countries.

When the parsnip extract is used as a food additive, the parsnip extract may be used directly or in combination with other foods or food ingredients, and may be used appropriately according to conventional methods. The amount of active ingredients mixed may be appropriately determined according to its intended use.

As another aspect for achieving the above objects, the present disclosure provides a pharmaceutical composition containing parsnip extract as an active ingredient for preventing or treating leaky gut syndrome or a metabolic disease.

The terms “parsnip,” “extract,” “active ingredient,” “leaky gut syndrome,” “metabolic disease,” “prevention,” and “composition” are as described above.

As used herein, the term “treatment” refers to any action that alleviates or beneficially alters the symptoms of leaky gut syndrome or a metabolic disease through administration of the composition of the present disclosure.

The pharmaceutical composition of the present disclosure may further include a pharmaceutically acceptable carrier. The pharmaceutical composition may further include, for example, a carrier for oral administration or a carrier for parenteral administration. The carrier for oral administration may include lactose, starch, cellulose derivatives, magnesium stearate, stearic acid, etc.

In addition, the carrier for parenteral administration may include water, suitable oils, saline, aqueous glucose, glycols, etc. The pharmaceutical composition may further include stabilizers and preservatives. Examples of suitable stabilizers include antioxidants such as sodium bisulfite, sodium sulfite, or ascorbic acid. Examples of suitable preservatives include benzalkonium chloride, methyl paraben, propyl paraben, and chlorobutanol.

The pharmaceutical composition of the present disclosure may be administered to mammals, including humans, via any route. For example, it may be administered orally or parenterally. Parenteral administration methods may include, but are not limited to, intravenous, intramuscular, intra-arterial, intracerebral, intramedullary, intrathecal, intracardiac, transdermal, subcutaneous, intraperitoneal, intranasal, gastrointestinal, topical, sublingual, or rectal administration.

The pharmaceutical composition of the present disclosure may be formulated as a preparation for oral or parenteral administration, according to the administration route described above. When formulated, the composition may be prepared using one or more buffers (e.g., saline or phosphate-buffered saline (PBS)), antioxidants, bacteriostatic agents, chelating agents (e.g., EDTA or glutathione), fillers, bulking agents, binders, adjuvants (e.g., aluminum hydroxide), suspending agents, thickening agents, humectants, disintegrants, surfactants, diluents, or excipients.

Solid preparations for oral administration include tablets, pills, powders, granules, liquids, gels, syrups, slurries, suspensions, capsules, etc. These solid preparations may be prepared by mixing the pharmaceutical composition of the present disclosure with at least one excipient, for example, starches (including corn starch, wheat starch, rice starch, potato starch, etc.), calcium carbonate, sucrose, lactose, dextrose, sorbitol, mannitol, xylitol, erythritol, maltitol, cellulose, methylcellulose, sodium carboxymethylcellulose, hydroxypropyl methylcellulose, or gelatin. For example, tablets or sugar-coated tablets may be obtained by blending the active ingredient with a solid excipient, grinding the mixture, adding suitable auxiliary agents thereto, and then processing the mixture into granule mixtures.

In addition to simple excipients, lubricants such as magnesium stearate and talc are also used. Liquid preparations for oral administration include suspensions, oral solutions, emulsions, and syrups. In addition to commonly used simple diluents such as water or liquid paraffin, various excipients, for example, humectants, sweeteners, flavoring agents, or preservatives may be included. Furthermore, in some cases, cross-linked polyvinylpyrrolidone, agar, alginate, or sodium alginate may be added as disintegrants, and the composition may further include anti-caking agents, lubricants, humectants, flavoring agents, emulsifiers, and preservatives.

When administered parenterally, the pharmaceutical composition of the present disclosure may be formulated with a suitable parenteral carrier into an injectable preparation, a transdermal preparation, or a nasal inhalation preparation according to methods known in the art. For injectable preparations, sterilization is required, and they are required to be protected from contamination by microorganisms such as bacteria and fungi. For injectable preparations, suitable carriers may include, but are not limited to, solvents or dispersion media including water, ethanol, polyols (e.g., glycerol, propylene glycol, and liquid polyethylene glycol), a mixture thereof, and/or plant oils. More preferably, suitable carriers include Hank's solution, Ringer's solution, PBS containing triethanolamine, sterile water for injection, or isotonic solutions such as 10% ethanol, 40% propylene glycol, and 5% dextrose. To protect the injectable preparations from microbial contamination, the composition may further include various antibacterial and antifungal agents such as parabens, chlorobutanol, phenol, sorbic acid, and thimerosal. Furthermore, in most cases, the injectable preparations may further include isotonic agents such as sugars or sodium chloride.

Transdermal preparations include forms such as ointments, creams, lotions, gels, topical solutions, pastes, liniments, and aerosols. Here, “transdermal administration” refers to the topical administration of the pharmaceutical composition to the skin, whereby an effective amount of the active ingredient contained in the pharmaceutical composition is delivered into the skin.

For inhalation preparations, the compositions used according to the present disclosure may be conveniently delivered in the form of an aerosol spray from a pressurized pack or nebulizer using a suitable propellant, for example, dichlorofluoromethane, trichlorofluoromethane, dichlorotetrafluoroethane, carbon dioxide, or other suitable gases. For pressurized aerosols, the dosage unit may be determined by providing a valve that delivers a metered dose. For example, gelatin capsules and cartridges used in inhalers or insufflators may be formulated to contain a powdered mixture of the compound and a suitable powdered carrier such as lactose or starch. Formulations for parenteral administration are described in well-known standard references in pharmaceutical chemistry, such as Remington's Pharmaceutical Sciences, 15th Edition, 1975, Mack Publishing Company, Easton, Pennsylvania, Chapter 87: Blaug, Seymour.

The pharmaceutical composition of the present disclosure is administered in a pharmaceutically effective amount. The term “pharmaceutically effective amount” refers to an amount sufficient to treat a disease at a reasonable benefit/risk ratio applicable to medical treatment and not causing side effects. The effective dose level may be determined according to factors including the patient's health condition, type and severity of the disease, drug activity, sensitivity to the drug, administration method, administration time, administration route and excretion rate, treatment duration, combination or concurrent use of other drugs, and other factors well known in the medical field. The dosage and frequency of administration do not in any way limit the scope of the present disclosure.

The pharmaceutical composition of the present disclosure may be used alone or in combination with surgery, radiation therapy, hormone therapy, chemotherapy, or biological response modifiers.

The pharmaceutical composition of the present disclosure may be administered to mammals such as mice, dogs, cats, cattle, horses, pigs, and humans, via various routes, with humans being preferred. All methods of administration are contemplated, including, but not limited to, oral, intravenous, intramuscular, or subcutaneous injection.

In another aspect for achieving the above objects, the present disclosure provides a quasi-drug composition containing parsnip extract as an active ingredient for preventing or improving leaky gut syndrome or a metabolic disease.

The terms “parsnip,” “extract,” “active ingredient,” “leaky gut syndrome,” “metabolic disease,” “prevention,” “improvement,” and “composition” are as described above.

As used herein, the “quasi-drug” refers to any article intended for use in humans or animals for the diagnosis, treatment, alleviation, management, or prevention of diseases, excluding instruments, machines, or devices, and any article intended to exert pharmacological effects on the structure or function of humans or animals, also excluding instruments, machines, or devices. Specific examples thereof include, but are not limited to, oral preparations. The formulation method, dosage, modes of use, and compositions of quasi-drugs may be appropriately selected from conventional techniques known in the art.

The quasi-drug composition of the present disclosure may further include, in addition to the above components, pharmaceutically acceptable carriers, excipients, or diluents, as needed. The pharmaceutically acceptable carriers, excipients, or diluents are not limited as long as they do not impair the effect of the present disclosure, and may include, for example, fillers, bulking agents, binders, humectants, disintegrants, surfactants, lubricants, sweeteners, flavoring agents, preservatives, etc.

In another aspect for achieving the above objects, the present disclosure provides a feed composition containing parsnip extract as an active ingredient for improving intestinal health, or preventing or improving leaky gut syndrome or a metabolic disease.

The terms “parsnip,” “extract,” “active ingredient,” “intestinal health,” “leaky gut syndrome,” “metabolic disease,” “prevention,” “improvement,” and “composition” are as described above.

As used herein, the term “feed” refers to any natural or artificial formulated diet, a single feeding, or components thereof, intended for consumption, ingestion, and digestion by animals, and may be prepared in various forms of feed known in the art. Specifically, examples thereof may include, but are not limited to, concentrated feeds, roughage, feed additives, feed supplements, nutritional supplements for pets, or special feeds.

The feed additives of the present disclosure correspond to auxiliary feeds under the Feed Management Act, and may additionally include mineral preparations such as sodium bicarbonate, bentonite, magnesium oxide, and complex minerals, mineral preparations, which are trace minerals such as zinc, copper, cobalt, and selenium, vitamins such as carotene, vitamin E, vitamins A, D, and E, nicotinic acid, and vitamin B complex, protected amino acids such as methionine and lysine, protected fatty acids such as fatty acid calcium salts, live bacteria such as probiotics (lactic acid bacteria), yeast cultures, and mold fermentations, and yeast agents.

Concentrated feeds include, but are not limited to, seed fruits including cereals such as wheat, oats, and corn, cereal bran, obtained as a by-product from refining grains, including rice bran, wheat bran, and barley bran, oil cakes obtained as by-products from oil extraction of beans, rapeseeds, sesame seeds, flaxseeds, coconuts, etc., residues such as residual starchy substances, which are the main components of starch residues remaining after starch extraction from sweet potatoes, potatoes, etc., animal-derived feeds such as fish meal, fish waste, fish solubles obtained by concentrating fresh liquid substances derived from fish, meat meal, blood meal, feather meal, skim milk powder, dried whey obtained by drying whey, which is the residue remaining when cheese is produced from milk or casein is produced from skim milk, yeast, chlorella, and seaweeds. Roughages include, but are not limited to, fresh forage such as wild grasses, pasture grasses, and green forage, root vegetables such as feed turnips, feed beets, and rutabagas, which are a type of turnip, silage obtained by packing fresh forage, green forage crops, grains, etc. into a silo and subjecting them to lactic acid fermentation, hay obtained by cutting and drying wild grasses or pasture grasses, straws from breeding crops, and leaves of leguminous plants. Special feeds include, but are not limited to, mineral feeds such as oyster shells and rock salt, urea feeds such as urea or a derivative thereof, including diureidoisobutane, feed additives, which are substances added in small amounts to compound feeds to supplement nutrients that are often deficient when only natural feed ingredients are blended or to improve feed storability, and dietary supplements.

The feed composition of the present disclosure may further include ingredients added to conventional feeds. Examples of ingredients added to such feeds may include cereal powders, meat powders, and legumes. In the foregoing, the cereal powders may include one or more selected from rice flour, wheat flour, barley flour, and corn flour. In the foregoing, the meat powders may include a powdered meat powder obtained by grinding one or more types of meat selected from chicken, beef, pork, and ostrich. In the foregoing, the legumes may include one or more selected from soybean, kidney bean, pea, and black bean.

The feed composition of the present disclosure may further include, in addition to the cereal powders, meat powders, and legumes mentioned above as components conventionally added to feeds, one or more selected from nutritional supplements and minerals to enhance the nutritional value of the feed, and one or more selected from antifungal agents, antioxidants, anticoagulants, emulsifiers, and binders to prevent deterioration of feed quality.

The feed or feed additive of the present disclosure may be applied to a wide variety of animal diets, including those of mammals, poultry, and fish.

In another embodiment for achieving the above objects, the present disclosure provides a method for preventing or treating leaky gut syndrome or a metabolic disease, comprising administering to a subject a composition containing parsnip extract as an active ingredient.

In another embodiment for achieving the above objects, the present disclosure provides a method for improving intestinal health, comprising administering to a subject a composition containing parsnip extract as an active ingredient.

In another embodiment for achieving the above objects, the present disclosure provides a use of a composition containing parsnip extract as an active ingredient, for the improvement of intestinal health.

In another embodiment for achieving the above objects, the present disclosure provides a use of a composition containing parsnip extract as an active ingredient, for the prevention or treatment of leaky gut syndrome or a metabolic disease.

The terms “parsnip,” “extract,” “active ingredient,” “composition,” “intestinal health,” “leaky gut syndrome,” “metabolic disease,” “prevention,” “improvement,” and “treatment” are as described above.

The subject may be mammals, specifically humans, cattle, sheep, goats, horses, pigs, dogs, cats, rabbits, rats, or mice, or non-mammals, such as fish or birds, but is not limited thereto.

### BRIEF DESCRIPTION OF THE DRAWINGS

FIG. 1 shows the results of measuring the prebiotic activity scores of beneficial intestinal bacteria upon administration of parsnip extract.

FIG. 2 shows the detection concentration of FITC-dextran (A) and area under the curve (B) upon administration of parsnip extract.

FIG. 3 shows the results confirming the efficacy of reducing the downregulation of intestinal permeability-related membrane protein expression upon administration of parsnip extract.

FIG. 4 shows the results of changes in body weight (a) and changes in body shape and body fat percentage (b) in mice upon administration of parsnip extract.

FIG. 5 shows the results of liver weight (a), fasting blood glucose (b), changes in blood glucose (c), and oral glucose tolerance test (OGTT) measurements (d) in mice upon administration of parsnip extract.

FIG. 6 shows the results of measuring ALT levels (a), AST levels (b), total cholesterol levels (c), and triglyceride levels (d) upon administration of parsnip extract.

FIG. 7 shows the results of H&E staining performed to confirm changes in liver tissue damage upon administration of parsnip extract.

FIG. 8 shows the results of H&E staining performed to confirm changes in epididymal fat size upon administration of parsnip extract.

FIG. 9 shows the results of measuring insulin levels (a), insulin resistance (b), and leptin levels (c) upon administration of parsnip extract.

FIG. 10 shows the results of electrophoresis to confirm for the expression level of lipid metabolism-related biomarker proteins upon administration of parsnip extract.

FIG. 11 shows the results of Bradford protein quantification to confirm the expression level of lipid metabolism-related biomarker proteins upon administration of parsnip extract.

### DETAILED DESCRIPTION

Hereinafter, the present disclosure will be described in more detail with reference to examples. However, these examples are intended to illustrate the present disclosure and are not intended to limit the scope of the present disclosure.

### Example 1: Preparation of Parsnip Extract

Parsnip roots were purchased from Yecheon, Gyeongbuk, Republic of Korea, and used. The parsnip roots were dried in a drying oven at 50° C., and then ground using a grinder to a particle size of 2.0 mm or less. Subsequently, water in an amount equivalent to 20 times the volume of 5 g of the sample was added, and hot water extract was obtained by reflux extraction at a temperature of 80° C. for 3 hours. The parsnip extract obtained by this method is hereinafter referred to as “parsnip root extract (PRE).”

### Experimental Example 1: Confirmation of Effect of Parsnip Extract on Improving Intestinal Environment

### 1.1. Experimental Method

### 1.1.1. Strains

The prebiotic activity score was measured as a method for evaluating the efficacy of the parsnip extract on the proliferation of beneficial intestinal bacteria. Lactobacillus bulgaricus MG5167, Lactobacillus gasseri MG4247, Bifidobacterium longum KCTC3421, and Bifidobacterium bifidum KCTC3440 strains, which were isolated from the human intestine, were used as the beneficial intestinal bacteria. Escherichia coli KCTC2441 was used as E. coli strain.

For cultivation, Lactobacillus strains were grown in MRS medium at pH 6.5±0.2 and 37° C. for 24 hours under facultative anaerobic conditions, and preserved by lyophilization or by freezing cell suspensions. Bifidobacteria strains, which are obligate anaerobes, were cultured in TSA medium supplemented with 5% sheep blood and 0.05% L-cysteine under anaerobic conditions at 37° C. for 48 to 72 hours. E. coli strains, which are facultative anaerobes, were cultured in TSA medium at 37° C. for 24 hours.

### 1.1.2. Prebiotic Activity Evaluation

M9 minimal broth (MB) supplemented with 2 g/L glucose, 0.015 g/L calcium chloride (CaCl₂)), and 0.5 g/L magnesium sulfate (MgSO₄) was used as the culture medium for prebiotic activity score analysis.

Specifically, the culture was performed by spreading colonies of each strain onto agar corresponding to the strain and incubating them in an incubator at 37° C. for 24 to 48 hours for primary culture. Thereafter, the colonies were transferred to 10 mL of liquid broth and cultured in an incubator at 37° C. for 24 to 48 hours for secondary culture. M9 medium was mixed with 5% (v/v) bacterial culture and either 5 mg/ml of the control (glucose) or the parsnip extract treatment group (1 mg/mL and 5 mg/mL concentration). The absorbance was measured at 600 nm using a microplate reader immediately after mixing (0 hour) and after 24 to 48 hours, and these values were applied to Equation 1 below to obtain a prebiotic activity score.

Here, as a positive control group, inulin, which is a prebiotic reported to improve enteric microbiota, was used at a concentration of 5 mg/mL.

$$Prebiotic activity score = \frac{\begin{matrix}Probiotic log CFU {mL}^{- 1} on the prebiotic at 24 h - \\ Probiotic log CFU {mL}^{- 1} on the prebiotic at 0 h\end{matrix}}{\begin{matrix}Probiotic log CFU {mL}^{- 1} on glucose at 24 h - \\ Probiotic log CFU {mL}^{- 1} on glucose at 0 h\end{matrix}} - \frac{\begin{matrix}Enteric log CFU {mL}^{- 1} on the prebiotic at 24 h - \\ Enteric log CFU {mL}^{- 1} on the prebiotic at 0 h\end{matrix}}{\begin{matrix}Enteric log CFU {mL}^{- 1} on glucose at 24 h - \\ Enteric log CFU {mL}^{- 1} on glucose at 0 h\end{matrix}} \qquad [ Equation 1 ]$$

### 1.1.3. pH Analysis

The pH change of the medium before and after cultivation was measured three times using a pH meter (Orion Star A211, Thermo Fisher Scientific), and the average values were used.

### 1.2. Confirmation of Effect on Prebiotic Activity

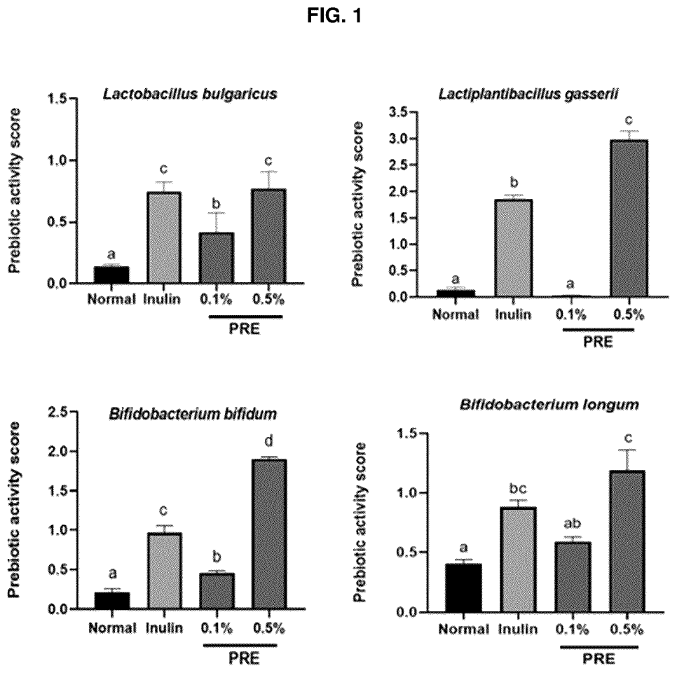

As a result of measuring the prebiotic activity scores of individual beneficial intestinal bacteria using parsnip extract, as shown in FIG. 1, it was confirmed that the evaluated strains, two Lactobacillus species and two Bifidobacterium species, exhibited significantly higher bacterial proliferative capacity at a 0.5% concentration of parsnip extract compared to the positive control group, inulin.

Specifically, administration of a 0.5% concentration of parsnip extract showed that the prebiotic activity of the beneficial bacteria, L. bulgaricus and L. gasseri strains, were approximately 1.2- and 1.6-fold higher, respectively, than that of the positive control group, inulin, and that for B. longum and B. bifidum strains, the prebiotic activity was approximately 1.3- and 2-fold higher, respectively, than that of the positive control group, inulin. These values were also 2.0- and 4.2-fold higher, respectively, than those observed with a 0.1% concentration of parsnip extract.

### 1.3. Confirmation of pH Change

As a result of measuring the pH of each strain upon administration of parsnip extract, as shown in Table 1 below, it was confirmed that the pH of the culture media for L. bulgaricus and L. gasseri strains treated with a 0.5% concentration of parsnip extract reduced from 6.81 and 6.80 before the reaction to 6.27 and 6.20 after the reaction, respectively, showing a decrease of approximately 0.6, and that the pH of the culture media for B. longum and B. bifidum strains treated with a 0.5% concentration of parsnip extract reduced from 6.62 and 6.81 before the reaction to 4.35 and 5.05 after the reaction, respectively, showing a decrease of approximately 1.8 to 2.3. This decrease was greater than the pH changes observed in the positive control group, inulin, before and after the reaction.

TABLE 1

| | Control | | Positive control | | PRE | | | |
|---|---|---|---|---|---|---|---|---|
| | (Normal) | | (Inulin) | | 0.1% | | 0.5% | |
| Incubation | Before | After | Before | After | Before | After | Before | After |
| L. bulgaricus | 6.76 ± | 6.68 ± | 6.80 ± | 6.34 ± | 6.81 ± | 6.44 ± | 6.81 ± | 6.27 ± |
| | 0.00 | 0.00 | 0.00 | 0.01 | 0.01 | 0.00 | 0.01 | 0.01 |
| L. gasseri | 6.80 ± | 6.68 ± | 6.83 ± | 6.44 ± | 6.81 ± | 6.68 ± | 6.80 ± | 6.20 ± |
| | 0.01 | 0.00 | 0.01 | 0.01 | 0.01 | 0.01 | 0.00 | 0.01 |
| B. longum | 6.64 ± | 6.25 ± | 6.62 ± | 4.53 ± | 6.55 ± | 5.91 ± | 6.62 ± | 4.35 ± |
| | 0.01 | 0.06 | 0.01 | 0.04 | 0.05 | 0.04 | 0.01 | 0.30 |
| B. bifidum | 6.84 ± | 6.68 ± | 6.86 ± | 6.22 ± | 6.78 ± | 5.59 ± | 6.81 | 5.05 ± |
| | 0.01 | 0.00 | 0.01 | 0.01 | 0.00 | 0.11 | 0.01 | 0.01 |

As a result, it was confirmed that parsnip extract lowered the pH toward a slight acidity four evaluated strains, L. bulgaricus, L. gasseri, B. longum, and B. bifidum, compared to the positive control group, inulin.

This suggests that short-chain fatty acids (SCFAs) were produced during the culture, and they are expected to confer beneficial effects on health, including serving as an energy source for intestinal epithelial cells, strengthening the immune system, and regulating metabolism. Furthermore, this suggests that maintaining a slightly acidic intestinal environment may help suppress the proliferation of harmful bacteria.

### Experimental Example 2. Confirmation of Effect of Parsnip Extract on Improving Leaky Gut Syndrome

### 2.1. Experimental Method

Forty-five 8-week-old male C57BL/6 mice were purchased from Orient Co., Ltd.

The experimental groups for the animal experiment were configured as follows:
Normal diet group (ND): Experimental group fed a normal diet (AlN-93G) and administered only sterile saline orally;
High-fat diet group (HF): Experimental group fed a high-fat (60% kcal fat) diet and administered only sterile saline orally;
Garcinia-treated group (GG): Experimental group fed a high-fat diet and administered 50 mg/kg of Garcinia cambogia extract orally;
50 mg/kg extract-treated group (PRE50): Experimental group fed a high-fat diet and administered 50 mg/kg of parsnip extract orally;
100 mg/kg extract=treated group (PRE100): Experimental group fed a high-fat diet and administered 100 mg/kg of parsnip extract orally.

Nine experimental animals were used in each group, and after an 8-week feeding period, intestinal immune-modulating efficacy was evaluated.

Thereafter, the efficacy of parsnip extract in improving leaky gut syndrome was evaluated using mice that were orally administered the parsnip extract for 8 weeks. The experimental animals were fasted for 6 hours prior to the experiment, after which the experiment was conducted. To confirm intestinal permeability, 400 mg/kg body weight of FITC-dextran dissolved in sterile saline was orally administered, and blood samples were collected from the tail vein at 0, 2, and 4 hours. The obtained 100 μL of blood was centrifuged (12,000×g, 10 min, 4° C.) to collect the supernatant, which was then used to measure absorbance with a microplate reader set at an excitation wavelength of 485 nm and an emission wavelength of 535 nm. FITC-dextran concentration was calculated using a standard curve and represented as the area under the curve (AUC) at each time point.

### 2.2. Experimental Results

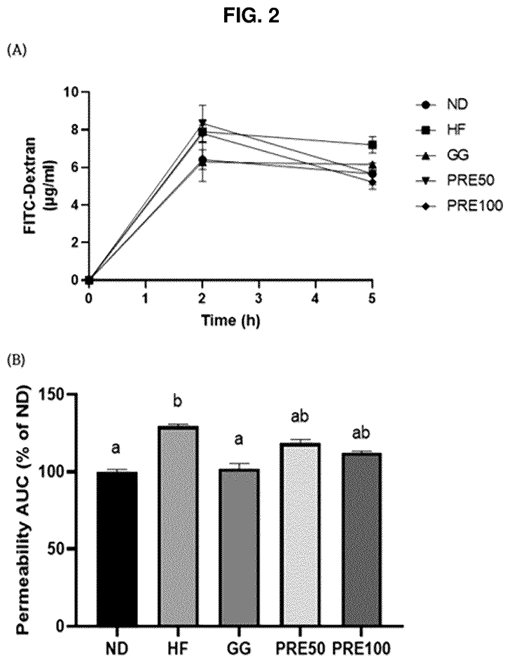

The results confirming the effects of parsnip extract on improving leaky gut syndrome are shown in FIG. 2. The concentrations of FITC-dextran detected at each time point were measured (FIG. 2A) and represented as the area under the curve (AUC) (FIG. 2B).

Specifically, according to the area under the curve measurement results of FITC-dextran, when the normal diet group (ND) was expressed as 100%, the high-fat diet group (HF) increased by approximately 129.4%, and in the positive control group, the Garcinia extract-treated group (GG), intestinal permeability was 101.96%, indicating suppression of intestinal permeability compared to the high-fat diet group despite the concurrent high-fat diet. Meanwhile, in the parsnip extract groups, AUC values were detected as 118.46% at a 50 mg/kg concentration and 112.25% at a 100 mg/kg concentration, indicating that the parsnip extract also suppressed intestinal permeability in a manner similar to the Garcinia extract-treated group, with higher doses showing greater suppression.

Therefore, it was confirmed that the parsnip extract of the present disclosure has an effect in improving leaky gut syndrome.

### Experimental Example 3. Confirmation of Effect of Parsnip Extract on Modulating Intestinal Permeability Regulating Membrane Proteins

### 3.1. Experimental Method

After conducting an 8-week experiment using the method of Experimental Example 2.1 above on the normal diet group, high-fat diet group, Garcinia-treated group (positive control group), PRE50 group, and PRE100 group, colon tissues were isolated. The isolated colon tissues were homogenized in lysis buffer containing protease inhibitors and centrifuged at 10,000×g for 5 minutes, and the supernatant was separated. The proteins in the supernatant were quantified using a Bradford method with a protein assay kit (TaKaRa Bradford Protein Assay Kit). The quantified proteins were electrophoresed using a 10% SDS-polyacrylamide gel (Bio-Rad, CA, USA) and transferred to a PVDF membrane (Bio-Rad, CA, USA). After blocking the nonspecific binding sites with a 5% skim milk solution (0.1% Tween 20 containing PBS, PBST), the membranes were incubated with primary antibody (ZO-1, Abcam) overnight at 4° C., followed by incubation with secondary antibody (anti-rabbit IgG linked with horseradish peroxidase, Cell Signaling Technology) for 1 hour at room temperature. Detection was then performed using streptavidin-horseradish peroxidase and a ChemiDoc XRS system (Bio-Rad, USA).

### 3.2. Confirmation of Efficacy of Parsnip Extract on Modulating Intestinal Permeability Regulating Membrane Proteins

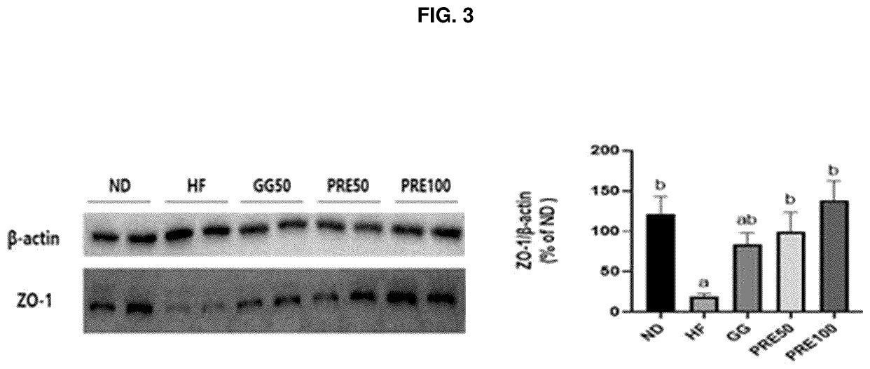

Changes in the expression levels of Zonula occludens-1 (ZO-1), a representative intestinal permeability regulating membrane protein, were analyzed in colon tissue. As a result, as shown in FIG. 3, it was confirmed that ZO-1 expression was reduced by approximately 80% in the high-fat diet-induced group compared with the normal diet group, whereas all parsnip extract-treated groups maintained expression levels comparable to those of the normal diet group. Specifically, it was confirmed that when the normal diet group was converted to a reference value of 100, the high-fat diet group had a value of 20.68, the positive control group, the Garcinia extract-treated group (GG) had a value of 68.99, and the parsnip extract groups had values of 81.90 at a 50 mg/kg concentration and 114.01 at a 100 mg/kg concentration.

These results suggest that parsnip extract consumption may improve intestinal permeability increased by a high-fat diet.

### Experimental Example 4. Confirmation of Effect of Parsnip Extract on Changes in Body Weight and Body Fat

### 4.1. Experimental Method

Forty-five 8-week-old male C57BL/6 mice were purchased from Orient Co., Ltd. Mice were housed at a temperature of 22±2° C. and a humidity of 40 to 60%, with the light and dark cycle adjusted to 12-hour intervals.

The mice were randomly divided into five groups (n=9). The normal diet group (ND) and high-fat diet group (HF) were orally administered only sterile saline, whereas the positive control group, PC (50 mg/kg Garcinia), the experimental groups, PRE50 (50 mg/kg parsnip extract) and PRE100 (100 mg/kg parsnip extract) were orally administered the respective compounds dissolved in sterile saline. AlN-93G (Dyets, Bethlehem, PA, USA) was used as the normal diet, and 60% kcal fat (D12492, Research Diets Inc., New Brunswick, NJ, USA) was used as the high-fat diet.

To confirm the anti-obesity effects of parsnip extract, body weight gain in experimental animals was measured weekly at 10 a.m. for eight weeks, starting from the first day of experimental feeding.

### 4.2. Confirmation of Effect of Parsnip Extract on Reducing Body Weight and Body Fat

To confirm the anti-obesity efficacy of parsnip extract, changes in body weight in the mouse experimental group established in Experimental Example 1.1 were monitored over the 8-week period, and the body fat mass of the experimental animals was measured at week 8.

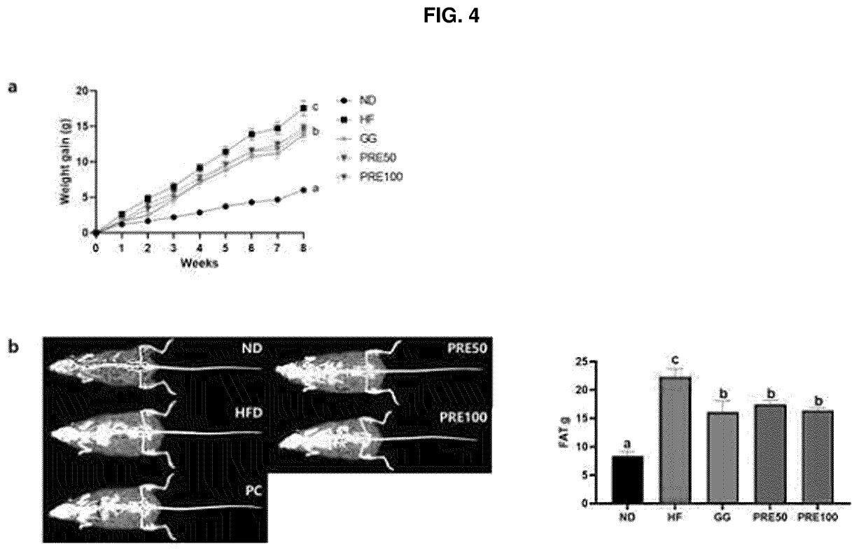

As a result, as shown in FIG. 4a, the normal control group (ND) gained 6.04 g of body weight over a total of 8 weeks, while the obese control group (HF) gained 18.07 g. In addition, the Garcinia-treated groups (positive control group, GG, PC) and the groups treated with parsnip extract at 50 mg/kg (PRE50) and 100 mg/kg (PRE100) while on a high-fat diet, showed final weight gains of 13.83 g, 14.71 g, and 14.32 g, respectively, demonstrating weight gain inhibition efficiency of 23.45%, 18.58%, and 20.74%, respectively, compared to the obese control group (HF). It was confirmed that all parsnip extract-treated groups showed a significant suppression of body weight gain compared to the obese control group (HF) (p<0.05).

In addition, at week 8 of the experimental diet, the overall body shape and body fat mass of the experimental mice were measured using dual-energy X-ray absorptiometry. As shown in FIG. 4b, the photographic results of the overall body shape confirmed that the red-colored area corresponding to fat was significantly reduced in the parsnip extract-treated groups compared to the obese control group (HF or HFD).

Consolidating the above results, the parsnip extract of the present disclosure was confirmed to have a significant effect on weight reduction and suppression of body fat mass increase during a high-fat diet. Based on these results, the following experiment was conducted.

### Experimental Example 5. Confirmation of Effect of Parsnip Extract on Tissue Weight Changes and Glucose Tolerance Regulation in Mice

### 5.1. Experimental Method

To evaluate the efficacy of parsnip extract in improving high-fat diet-induced glucose intolerance, the animals were fasted for 15 hours prior to the experiment, after which an oral glucose tolerance test (OGTT) was conducted. Glucose solution was orally administered at 2 g/kg, and blood samples were collected from the tail vein at 0, 30, 60, 90, and 120 minutes. Blood glucose levels were then measured using a glucometer.

Furthermore, after completion of the blood glucose measurement experiment, experimental animals were fasted for 12 hours, anesthetized with isoflurane, and the abdomen and thoracic cavity were incised to excise the liver tissue from each experimental group. The excised tissues were rinsed with saline to remove blood and debris, and the weight was measured.

### 5.2. Confirmation of Effect of Parsnip Extract on Tissue Weight Changes and Glucose Tolerance Regulation

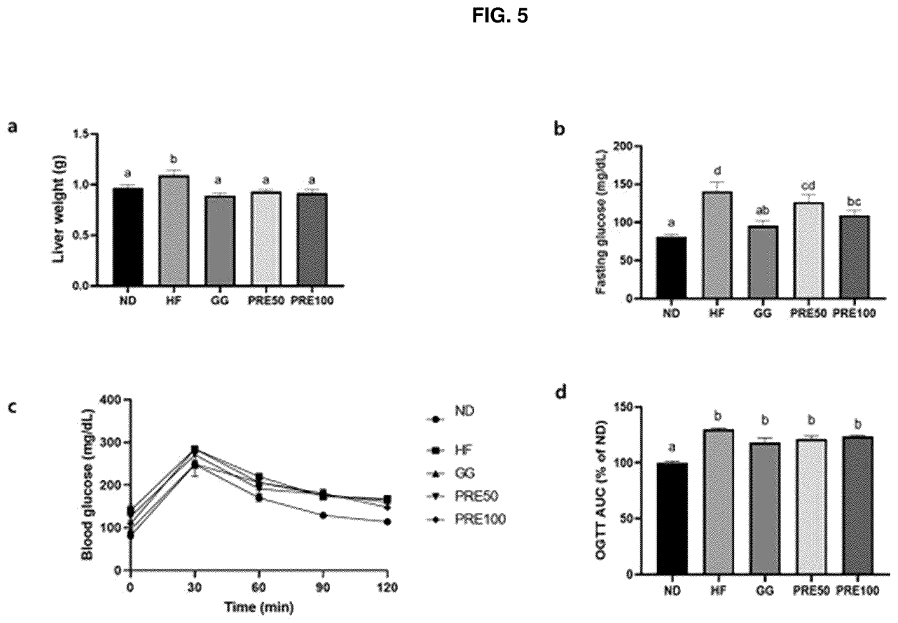

As a result of confirming the effect of parsnip extract administration on liver weight change, as shown in FIG. 5a, it was confirmed that the liver weights excised from the experimental animals were reduced by 18.02% 18.02%, 14.68%, and 16.08% in the Garcinia positive control (GG), PRE50, and PRE100 experimental groups, respectively, compared to the obese control group (HF), and the increase in liver weight was significantly suppressed in the PRE50 and PRE100 groups compared to the obese control group (p<0.05). These results confirmed that the administration of parsnip extract may suppress high-fat diet-induced fatty liver symptoms.

In addition, as a result of measuring fasting blood glucose to evaluate the efficacy against glucose intolerance, one of the metabolic diseases caused by obesity, as shown in FIG. 5b, it was confirmed that the parsnip extract-treated groups showed lower fasting blood glucose levels compared to the high-fat diet group (HF). Furthermore, as a result of measuring blood glucose changes over 120 minutes, as shown in FIG. 5c, it was confirmed that the parsnip extract-treated group showed lower blood glucose levels compared to the high-fat diet group. As a result of measuring the OGTT and calculating the area, as shown in FIG. 5d, it was confirmed that the parsnip extract-treated group showed reductions of 8.89% and 6.42%, respectively, compared to the obese control group.

Consolidating the above results, the parsnip extract of the present disclosure was confirmed to have a significant effect on liver weight and was also observed to alleviate glucose intolerance caused by obesity. Therefore, it is expected that the parsnip extract may exhibit anti-obesity effects.

### Experimental Example 6. Confirmation of Effect of Parsnip Extract on Obesity and Metabolic Disease in Mice

### 6.1. Experimental Method

After completion of the blood glucose measurement experiment in Example 5.1, experimental animals were fasted for 12 hours, anesthetized with isoflurane, and the abdomen and thoracic cavity were incised. Blood collected from the portal vein was allowed to coagulate, and serum was isolated. This was used to analyze the concentrations of liver function-related enzymes and serum lipid-related biomarkers.

Subsequently, alanine aminotransferase (ALT), aspartate aminotransferase (AST), and total cholesterol were analyzed using an enzyme-linked immunosorbent assay kit (Cell Biolabs, Inc., Beverly, MA, USA) and triglycerides were analyzed using a kit from Cayman Chemical (Ann Arbor, MI, USA). Liver and epididymal fat tissues were subjected to H&E staining to analyze the fat and extent of damage in the tissue. Blood collected from the portal vein was allowed to coagulate, and the serum was separated to measure insulin levels. To assess insulin resistance as a marker of metabolic diseases, an Insulin Mouse ELISA Kit (Invitrogen) was used, and to analyze lipid metabolism, leptin was analyzed using a kit (R&D Systems). Through this, the anti-obesity and metabolic disease-suppressing efficacy was physiologically observed.

### 6.2. Confirmation of Effect of Parsnip Extract on Changes in Mouse Serum Liver Function-Related Enzyme Levels

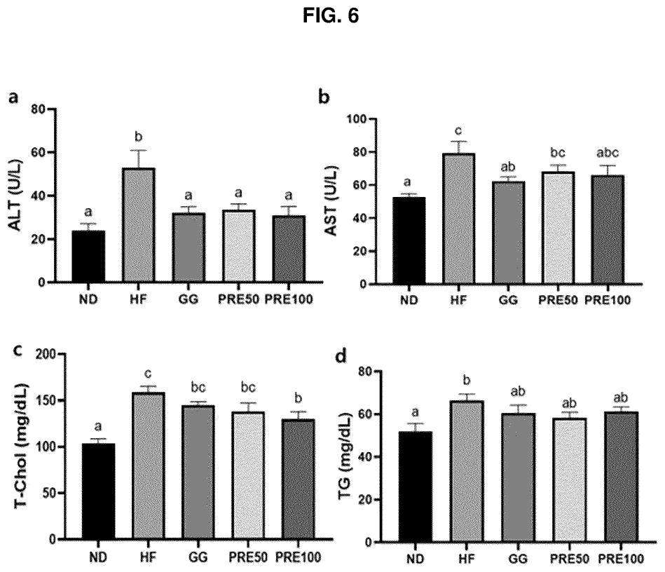

The results of confirming serum concentration of ALT and AST, which are used as indicators to infer the presence of liver disease and are biomarkers of fatty liver, a metabolic disease caused by obesity), are shown in FIGS. 6a and 6b.

Specifically, it was confirmed that ALT levels in the positive control group (GG) and the parsnip extract-treated groups (PRE50 and PRE100) were reduced by approximately 39.39%, 36.69%, and 41.76%, respectively, compared to the obese control group (HF), indicating that ALT levels were reduced in a concentration-dependent manner with parsnip extract (p<0.05). Furthermore, it was confirmed that AST levels were also significantly reduced in the parsnip extract-treated groups (PRE50 and PRE100) by approximately 13.86% and 16.61%, respectively, compared to the obese control group (HF) (p<0.05), indicating that parsnip extract administration had the efficacy of alleviating ALT and AST levels.

### 6.3. Confirmation of Effect of Parsnip Extract on Changes in Neutral Fat and Cholesterol Concentrations in Mice

Subsequently, serum triglyceride and total cholesterol concentrations were measured in the experimental groups. As a result, as shown in FIGS. 6c and 6d, both total cholesterol (T-Chol) and triglyceride (TG) levels increased in the high-fat diet-induced obese control group (HF), compared to the normal diet group (ND).

In contrast, it was confirmed that when parsnip extract was administered, total cholesterol levels were significantly reduced compared to the obese control group (p<0.05), and the levels in the PRE50 and PRE100 groups were reduced by approximately 13.06% and 18.10%, respectively, compared to the obese control group. It was confirmed that similarly, triglyceride levels were also significantly reduced in the parsnip extract-treated groups (PRE50 and PRE100) by approximately 12.22% and 7.89%, respectively, compared to the obese control group (HF) (p<0.05), indicating that parsnip extract administration had the efficacy in suppressing increase in both serum total cholesterol concentration and triglyceride.

### 6.4. Confirmation of Effect of Parsnip Extract on Changes in Liver Tissue Damage and Epididymal Fat Size in Mice

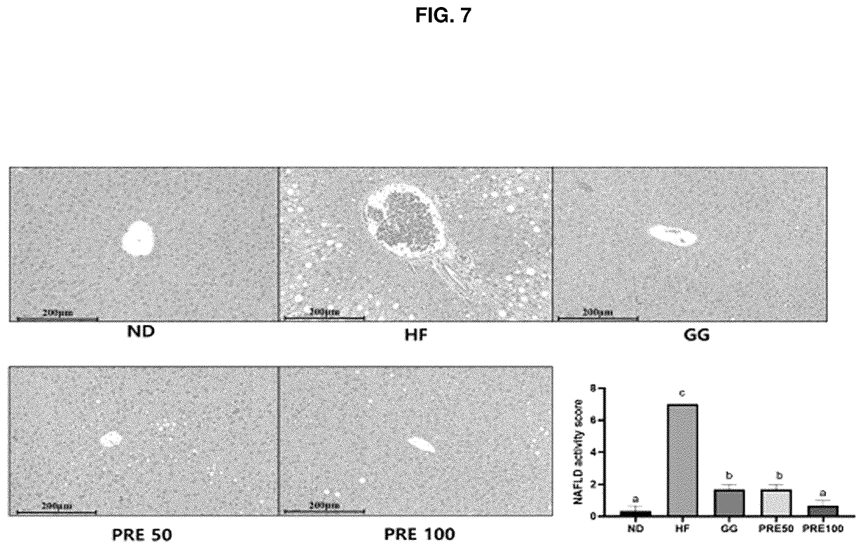

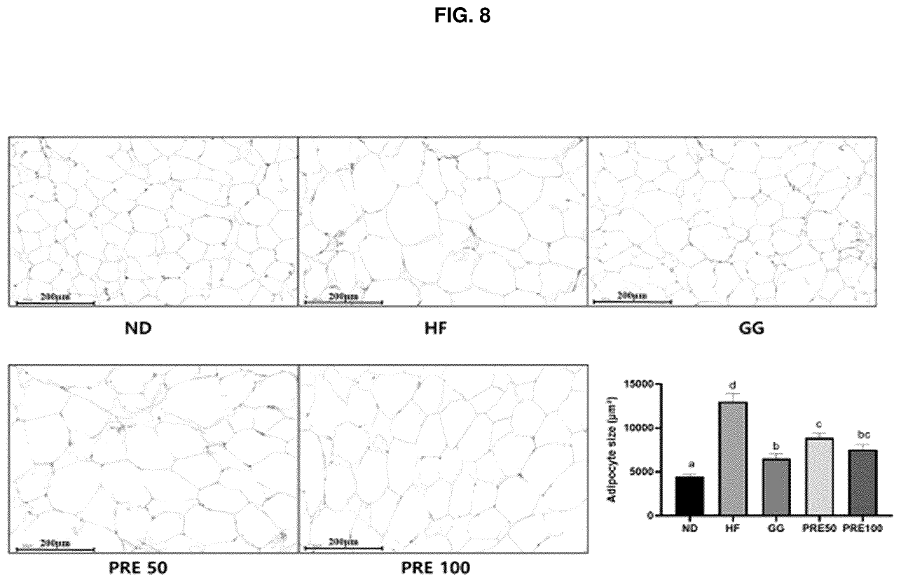

Liver tissues and epididymal fat excised from experimental animals were subjected to hematoxylin and eosin (H&E) staining to measure the extent of liver tissue damage and epididymal fat size, and the results are shown in FIGS. 7 and 8.

Liver H&E staining was scored based on NAFLD score, depending on the extent of liver damage. As a result, as shown in FIG. 7, it was confirmed that in the high-fat diet-induced obese control group (HF), liver damage was increased compared to the normal diet group (ND). In contrast, it was confirmed that liver damage and inflammation were significantly reduced in the parsnip extract-treated groups, PRE50 and PRE100 experimental groups, by approximately 76.19% and 90.48%, respectively, compared to the obese control group (HF).

Also, it was confirmed that similarly, as the H&E staining results of the epididymal fat, as shown in FIG. 8, the fat size was increased in the high-fat diet-induced obese control group (HF) compared to the normal diet group (ND), while the administration of parsnip extract significantly suppressed the increase in an adipocyte size (p<0.05). The vales were reduced by approximately 31.84% and 41.85%, respectively, in the PRE50 and PRE100 experimental groups, compared to the obese control group (HF).

### 6.5. Confirmation of Effect of Parsnip Extract on Insulin Resistance and Lipid Metabolism in Mice

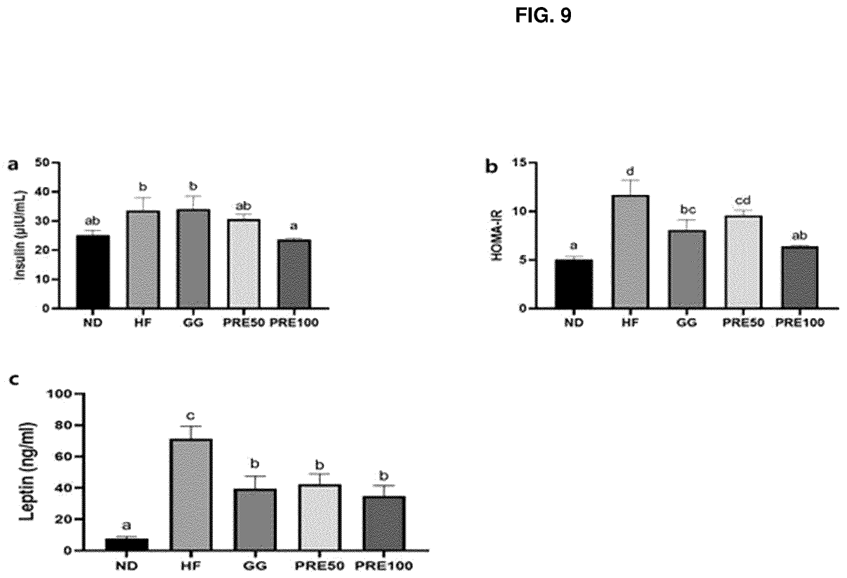

Blood collected from the portal vein of the experimental animals was allowed to coagulate, and the serum was separated to measure serum insulin and homeostasis model assessment of insulin resistance (HOMA-IR), which are indicators of metabolic diseases. The results are shown in FIGS. 9a and 9b.

It was confirmed that for insulin, the concentrations in the parsnip extract-treated groups (PRE50 and PRE100) were significantly reduced by approximately 8.86% and 29.70%, respectively, compared to the obese control group (HF) (p<0.05). It was confirmed that similarly, for HOMA-IR, the concentrations in PRE50 and PRE100 groups were also reduced by approximately 18.06% and 45.38%, respectively, compared to the obese control group (p<0.05). This decrease was dependent on the concentration of the parsnip extract, confirming that parsnip extract had the efficacy in alleviating both insulin and HOMA-IR levels.

In addition, as a result of confirming the serum concentration of hormone Leptin, a hormone closely related to fat metabolism, as shown in FIG. 9c, it was confirmed that the serum concentration levels of the hormone Leptin were reduced in a concentration-dependent manner with parsnip extract in the parsnip extract-treated groups, PRE50 and PRE100 experimental groups, compared to the high-fat diet-induced obese control group (HF) (p<0.05). The levels were reduced by approximately 40.56% and 50.99%, respectively, compared to the obese control group.

Consolidating the above results, it was confirmed that the administration of the parsnip extract of the present disclosure reduced insulin and insulin resistance levels, which are indicators of metabolic disease, and reduced the levels of leptin, which is a hormone associated with obesity, in a concentration-dependent manner, demonstrating that the parsnip extract may exert in anti-obesity efficacy and improve related metabolic diseases.

### 6.6. Confirmation of Effect of Parsnip Extract on Expression of Obesity-Related Proteins in Liver

After sacrificing experimental animals from the normal diet group, obese control group, positive control group, PRE50, and PRE100 groups, liver tissue was isolated. The isolated liver tissues were homogenized in lysis buffer containing protease inhibitors and centrifuged at 10,000×g for 5 minutes, and the supernatant was separated. The proteins in the supernatant were quantified using a Bradford method with a protein assay kit (TaKaRa Bradford Protein Assay Kit). The quantified proteins were electrophoresed using a 10% SDS-polyacrylamide gel (Bio-Rad, CA, USA) and transferred to a PVDF membrane (Bio-Rad, CA, USA). After blocking the nonspecific binding sites with a 5% skim milk solution (0.1% Tween 20 containing PBS, PBST), the membranes were incubated with primary antibody (ACC/CHREBP/FAS, Abcam; CEBPα/G6PD/SREBP1, Cell Signaling) overnight at 4° C., followed by incubation with secondary antibody (anti-rabbit IgG or anti-mouse IgG linked with horseradish peroxidase, Cell Signaling Technology) for 1 hour at room temperature. Detection was then performed using streptavidin-horseradish peroxidase and a ChemiDoc XRS system (Bio-Rad, USA).

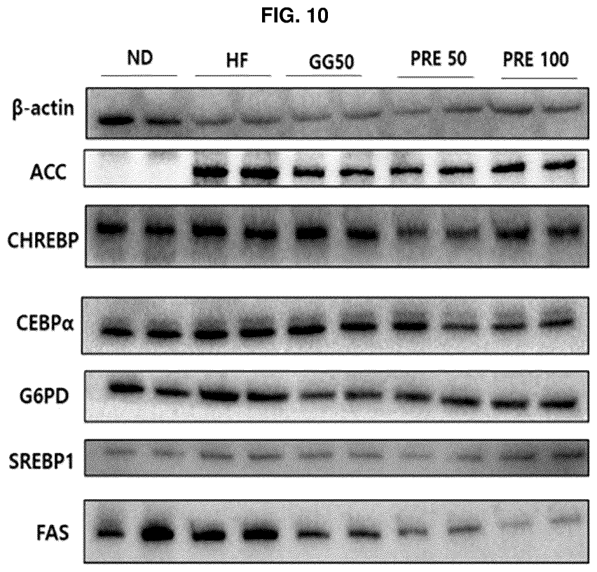

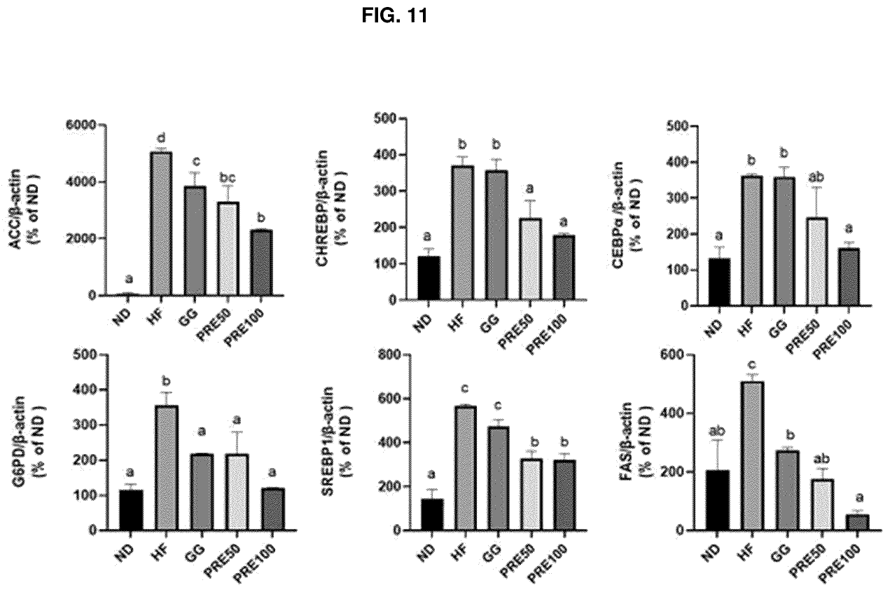

The results of analyzing the expression levels of biomarkers involved in lipid metabolism (ACC, CHREBP, CEBPα, G6PD, SREBP1, FAS) in liver tissue isolated from the normal diet group, obese control group, positive control group, PRE50 group, and PRE100 group are shown in FIGS. 10 and 11.

Specifically, as a result of the analysis of protein expression of fatty acid synthase (FAS) and acetyl-CoA carboxylase (ACC), enzymes involved in lipid metabolism, it was confirmed that high-fat diet (HF) administration increased the expression of these enzymes, whereas parsnip extract administration (PRE50 and PRE100 experimental groups) reduced the expression of FAS and ACC.

In addition, it was confirmed that similarly, for sterol regulatory element-binding protein 1 (SREBP1), a transcription factor regulating lipid metabolism, SREBP1 expression was increased by high-fat diet administration (HF), however, when parsnip extract was administered (PRE50 and PRE100 experimental groups), the increased SREBP1 expression was reduced.

As a result of the analysis of protein expression of carbohydrate response element binding protein (ChREBP) and glucose-6-phosphate dehydrogenase (G6PD), which promote lipogenesis, it was confirmed that the expression of ChREBP and G6PD was increased by high-fat diet administration, but in the parsnip extract-treated PRE50 and PRE100 experimental groups, the increased expression of ChREBP and G6PD was significantly decreased to the level of the normal diet group (ND).

Finally, as a result of the analysis of changes in protein expression of CCAAT/enhancer-binding protein alpha (CEBPα), a protein associated with lipolysis, it was confirmed that the expression of CEBPα was increased by high-fat diet administration, but in the parsnip extract-treated PRE50 and PRE100 experimental groups, the increased expression of CEBPα was reduced to normal levels.

The composition containing parsnip extract as an active ingredient according to the present disclosure may reduce the downregulation of tight junction protein expression, promote the proliferation of beneficial intestinal bacteria, improve the intestinal environment such as maintaining a slightly acidic intestinal environment, and have effects such as reducing intestinal permeability. Therefore, the composition may improve intestinal health and prevent, treat, or improve leaky gut syndrome, and may be used as a material for foods, feeds, pharmaceuticals, or quasi-drugs.

Additionally, the composition containing parsnip extract as an active ingredient according to the present may have body fat reduction and weight loss activities, reduce the protein expression of lipid metabolism-related factors such as ACC, FAS, SREBP1, ChREBP, G6PD, or C/EBPα, and have effects such as reducing liver weight, reducing blood sugar, reducing ALT or AST levels, reducing insulin resistance, and reducing extent of liver damage. Therefore, the composition may prevent, treat, or improve obesity or a metabolic disease, and may be used as a material for foods, feeds, pharmaceuticals, or quasi-drugs.

From the above description, those skilled in the art to which the present disclosure pertains will be able to understand that the present disclosure can be implemented in other specific forms without changing its technical spirit or essential features. In this regard, it should be understood that the embodiments described above are illustrative in all respects and not restrictive. The scope of the present disclosure should be interpreted as encompassing all modifications or variations derived from the meaning and scope of the claims set forth below, as well as their equivalents, rather than being limited by the foregoing detailed description.

1.-34. (canceled)

35. A method for improving or treating leaky gut syndrome or a metabolic disease, comprising:
administering to a subject in need thereof a composition comprising parsnip extract.

36. The method of claim 35, wherein the parsnip extract is obtained by extraction with water, a lower alcohol solvent having 1 to 4 carbon atoms, or a mixed solvent thereof.

37. The method of claim 35, wherein the improvement in intestinal health is an improvement in intestinal function.

38. The method of claim 35, wherein the composition reduces the downregulation of tight junction protein expression.

39. The method of claim 35, wherein the tight junction protein is Zonula occludens-1 (ZO-1).

40. The method of claim 35, wherein the composition improves an intestinal environment.

41. The method of claim 35, wherein the composition promotes the proliferation of beneficial intestinal bacteria.

42. The method of claim 41, wherein the beneficial intestinal bacteria are one or more selected from the group consisting of Lactobacillus bulgaricus, Lactobacillus gasseri, Bifidobacterium bifidum, and Bifidobacterium longum.

43. The method of claim 35, wherein the composition maintains the intestinal environment at a slightly acidic level.

44. The method of claim 35, wherein the composition reduces intestinal permeability.

45. The method of claim 35, wherein the metabolic disease is one selected from the group consisting of obesity, diabetes, hyperlipidemia, hypertension, hypercholesterolemia, hyperinsulinemia, arteriosclerosis, dyslipidemia, hypertriglyceridemia, liver disease, fatty liver, stroke, myocardial infarction, cardiovascular disease, hyperglycemia, and insulin resistance.

46. The method of claim 45, wherein the fatty liver disease is non-alcoholic fatty liver disease.

47. The method of claim 46, wherein the non-alcoholic fatty liver disease is selected from the group consisting of simple fatty liver disease, nutritional fatty liver disease, starvation-induced liver disease, obesity-related fatty liver disease, diabetic fatty liver disease, steatohepatitis, liver fibrosis, and cirrhosis.

48. The method of claim 35, wherein the composition suppresses lipid accumulation.

49. The method of claim 35, wherein the composition reduces serum levels of alanine aminotransferase (ALT) or aspartate aminotransferase (AST).

50. The method of claim 35, wherein the composition suppresses the expression of one or more proteins selected from the group consisting of ACC, CHREBP, CEBPα, G6PD, SREBP1, and FAS.

51. The method of claim 35, wherein the composition is a food composition.

52. The method of claim 35, wherein the composition is a pharmaceutical composition.

53. The method of claim 35, wherein the composition is a quasi-drug composition.

54. The method of claim 35, wherein the composition is a feed composition.
